(19) United States
(12) Patent Application Publication
(10) Pub. No.: US 2013/0217908 A1
(43) Pub. Date: Aug. 22, 2013

(54) STABILISATION OF RADIOPHARMACEUTICAL PRECURSORS

(71) Applicants: Tone Hauk Fritzell, Oslo (NO); Torild Wickstrom, Oslo (NO)

(72) Inventors: Tone Hauk Fritzell, Oslo (NO); Torild Wickstrom, Oslo (NO)

(73) Assignee: GE HEALTHCARE LIMITED, BUCKINGHAMSHIRE (GB)

(21) Appl. No.: 13/879,649

(22) PCT Filed: Oct. 28, 2011
(86) PCT No.: PCT/EP2011/068954

§ 371 Date: Apr. 16, 2013

Related U.S. Application Data
(60) Provisional application No. 61/407,470, filed on Oct. 28, 2010.

(51) Int. Cl. C07C 309/66

(52) U.S. Cl. CPC C07C 309/66
USPC 558/54

(57) ABSTRACT

The invention relates to a method for improving stability of radiopharmaceutical precursors, and in particular protected amino acid derivatives which are used as precursors for production of radiolabelled amino acids for use in in vivo imaging procedures such as positron emission tomography (PET). The invention further includes compositions comprising a radiopharmaceutical precursor and a drying agent, and cassettes for automated synthesis apparatus comprising the same.

The invention relates to a method for improving stability of radiopharmaceutical precursors, and in particular protected amino acid derivatives which are used as precursors for production of radiolabelled amino acids for use in in vivo imaging procedures such as positron emission tomography (PET). The invention further includes compositions of radiopharmaceutical precursors, and cassettes for automated synthesis apparatus comprising the same.

PET is effective in diagnosing a variety of diseases including heart diseases and cancer. Nuclear medicine imaging methods such as PET involve administering an agent labelled with a suitable radioisotope (a “radiopharmaceutical”) to a patient, followed by detecting γ-rays emitted directly or indirectly from the agent. These imaging methods are advantageous over other in vivo imaging methods in that as well as being highly specific and sensitive to diseases, they also provide information on the functionality of lesions. For example, the PET radiopharmaceutical [¹⁸F]2-fluoro-2-deoxy-D-glucose ([¹⁸F]FDG) concentrates in areas of glucose metabolism, thereby making it possible to specifically detect tumours in which glucose metabolism is enhanced. Nuclear medicine examination is performed by tracing a distribution of an administered radiopharmaceutical, and data obtained therefrom vary depending on nature of the radiopharmaceutical. Thus, different radiopharmaceuticals have been developed for a variety of applications, e.g. tumour diagnostic agents, bloodstream diagnostic agents and receptor mapping agents.

In recent years, a series of radioactive halogen-labelled amino acid compounds including [¹⁸F]1-amino-3-fluorocyclobutanecarboxylic acid ([¹⁸F]FACBC) have been designed as novel radiopharmaceuticals. [¹⁸F]FACBC is considered to be effective as a diagnostic agent for highly proliferative tumours, because it has a property of being taken up specifically by amino acid transporters. EP1978015(A1) describes precursors and processes for producing [¹⁸F]FACBC, wherein the process includes a step of adding radioactive fluorine to the precursor 1-(N-(t-butoxycarbonyl)amino)-3-[((trifluoromethyl)sulfonyl)oxy]-cyclobutane-1-carboxylic acid ethyl ester. The process further includes a step of deprotecting the compound to which radioactive fluoride has been added.

The precursor for [¹⁸F]FACBC, i.e. the compound 1-(N-(t-butoxycarbonyl)amino)-3-[((trifluoromethyl)sulfonyl)oxy]-cyclobutane-1-carboxylic acid ethyl ester is herein referred to as “FACBC triflate precursor”.

Protected amino acid derivatives, including non-radiolabelled amino acid derivatives such as the FACBC triflate precursor used in the preparation of [¹⁸F]FACBC, are unstable. The FACBC triflate precursor is neither stable in solution nor as a solid product for storage at room temperature. It is currently supplied as a solid and needs to be stored at sub-ambient temperatures, such as e.g. 5° C., to ensure stability over a reasonable period. This is an issue in the provision of a so-called “cassette” for use in the preparation of [¹⁸F]FACBC on an automated radiosynthesis system such as Fastlab™. This is because the FACBC triflate precursor has to be stored cold and separate from the other reagents and needs to be assembled into the cassette by the operator prior to running the radiolabelling process. Ideally, all of the reagents including the FACBC triflate precursor should be provided on one pre-assembled cassette suitable for room temperature storage.

Therefore, there is a need for a method to improve stability of non-radiolabelled amino acid derivatives, such as precursors for the radiosynthesis of [¹⁸F] FACBC, to improve shelf-life and preferably allow storage at ambient temperature, for example in the same package as the other reagents or as part of a pre-assembled cassette.

It has now surprisingly been found that by storing a non-radiolabelled amino acid derivative, such as a precursor for radiosynthesis of [¹⁸F] FACBC, in the presence of a drying agent, the stability is improved.

Therefore, in a first aspect the invention provides a method for improving the stability of a radiopharmaceutical precursor comprising storing said radiopharmaceutical precursor in the presence of a drying agent.

The “drying agent”, which can also be referred to as a desiccant, useful in the method of the invention is a substance that induces or sustains a state of dryness in its local vicinity working through absorption or adsorption of water. It hence removes excessive humidity that would degrade or destroy products, such as the pharmaceutical precursor. The drying agent used in the current invention is an inert material. It should not be a powder, and not be soluble in water or normal organic solvents. Suitable drying agents for use in the present invention are well-known to those of skill in the art. A preferred drying agent is a molecular sieve or silica gel, and is most preferably silica gel. Silica gel can be in the form of granules or beads, and is preferably in the form of granules. Preferred types are molecular sieve <3 mm with pore size 4A and silica gel granules <3 mm.

The amount of drying agent used must be sufficient to dry, and keep dry, the precursor. Hence, the drying agent absorbs or adsorbs water and will keep the precursor dry and stable when in presence of the drying agent. The weight ratio of drying agent:precursor is e.g. between 12:1 and 1:1, such as between 8:1 and 3:1, and preferably about 4:1.

By the term “in the presence of a drying agent” it is meant that the precursor is mixed with the drying agent, it is packed with the drying agent, or a composition is prepared comprising the precursor and a drying agent.

The term “radiopharmaceutical precursor” as used herein means a non-radioactive compound which may be radiolabelled with a suitable source of a radiolabel to prepare a radiopharmaceutical. In particular, the radiopharmaceutical precursor of the present invention is one that is unstable due to its hygroscopic properties. Radiolabels contemplated in the context of the present invention are suitable for imaging using PET or single-photon emission tomography (SPECT), e.g. ¹¹C or ¹⁸F for PET, and ¹²³I or ^{99m}Tc for SPECT. Methods for the synthesis of radiopharmaceuticals comprising these radiolabels are well-known to those of skill in the art, for example as described in chapters 5, 6, 10 and 13 of the “Handbook of radiopharmaceuticals: radiochemistry and applications” (by Michael J. Welch and Carol S. Redvanly; 2003 Wiley). In a preferred embodiment the radiopharmaceutical precursor is suitable for radiolabelling with [¹⁸F]. Preferably, the precursor includes a moiety suitable for nucleophilic substitution with the radiolabel, e.g. with [¹⁸F].

In one embodiment the radiopharmaceutical precursor is a non-radiolabelled amino acid derivative suitable for use as a precursor in synthesis of a radiolabelled amino acid derivative for PET or SPECT imaging. Such a non-radiolabelled amino acid derivative would include a moiety suitable for nucleophilic substitution with a radiolabel and the amino acid functional groups are optionally, but preferably, protected with suitable protective groups.

In a preferred embodiment, the radiopharmaceutical precursor is a cyclobutane-based amino acid derivative. More preferably, the radiopharmaceutical precursor is a compound of formula I:

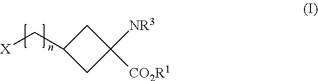

wherein,
n is an integer of 0 or of 1 to 4;
R¹ represents a protective group for the carboxylic acid function of the amino acid and is preferably a short alkyl group preferably selected from methyl, ethyl, 1-propyl or isopropyl substituent, and is preferably an ethyl substituent;
X is a moiety suitable for nucleophilic substitution or comprises a chelator; and,
R³ is a protective group for the amino function of the amino acid and is preferably selected from the group consisting of a t-butoxycarbonyl group, an allyloxycarbonyl group, a phthalimide group and N-benzylideneamine substituent, and is preferably a t-butoxycarbonyl group. Alternatively, the group NR³ can represent a nitrogen-containing heterocycle, preferably comprising a cyclic imide as described in WO 2006/126410. The term “cyclic imide” refers to a residue formed by removal of a hydrogen from a compound represented by the general formula Ia: R⁴—CONHCO—R⁵ wherein R⁴ and R⁵ bind to each other to form a ring. Accordingly, the nitrogen atom shown in the general formula Ia is bound to the carbon atom at position 1 of the cyclobutane ring of formula I. In formula Ia, R⁴ and R⁵ are preferably a carbon or sulfur atom and preferably form a 5-membered cyclic imide by linkage to each other. R⁴ and R⁵ may optionally have a substituent as long as they bind to each other. A preferred cyclic imide substituent is selected from a carbocyclic dicarboximide, saturated aliphatic dicarboximide and an unsaturated aliphatic dicarboximide. Examples of compounds wherein R³ is a cyclic imide are described in US 2009/0105489. A most preferred cyclic imide is the group:

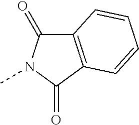

wherein the dotted line represents the point of connection to the rest of formula I.

The value of n may vary depending on the kinds of radioactive halogen-labelled amino acid compounds to be finally produced. For example, when the compound to be finally produced is a compound in which a halogen is directly bound to the cyclobutane ring (e.g. [¹⁸F] FACBC), n is 0, while when the compound to be finally produced is a compound in which a halogen is bound to the cyclobutane ring via a methylene chain, such as [¹⁸F] 1-amino-3-fluoromethylcyclobutanecarboxylic acid, n is 1.

The term “moiety suitable for nucleophilic substitution” represents a leaving group, selected from a halogen substituent and a group represented by —OR², wherein R² is selected from the group consisting of straight-chain or branched-chain haloalkylsulfonic acid substituents with one to 10 carbon atoms, trialkylstannyl substituents with 3 to 12 carbon atoms, fluorosulfonic acid substituents and aromatic sulfonic acid substituents. R² is preferably a substituent selected from the group consisting of toluenesulfonic acid substituent, nitrobenzenesulfonic acid substituent, benzenesulfonic acid substituent, trifluoromethanesulfonic acid substituent, fluorosulfonic acid substituent, perfluoroalkylsulfonic acid substituent, trimethylstannyl substituent and triethylstannyl substituent. When X is a halogen substituent, a bromo or chloro substituent can be preferably used. Most preferably, n is 0 and X is trifluoromethanesulfonate.

The term “chelator” refers to an organic compound capable of forming coordinate bonds with a metal ion through two or more donor atoms. The metal ion is one which is suitable for in vivo imaging by PET or SPECT. The term “comprises a chelator” encompasses the chelator alone and also the chelator with a bivalent linker present between it and the rest of the compound of formula I. Where present, the bivalent linker preferably comprises 1-10, most preferably 2-6, linker groups selected from —C(═O)—; —CH₂—; —NH—; —NHC(═O)—; —C(═O)NH—; and, —CH₂—O—CH₂—. In a typical chelator suitable in the context of the present invention 2-6, and preferably 2-4, metal donor atoms are arranged such that 5- or 6-membered chelate rings result (by having a non-coordinating backbone of either carbon atoms or non-coordinating heteroatoms linking the metal donor atoms). Examples of donor atom types which bind well to metal ions as part of chelating agents are: amines, thiols, amides, oximes, and phosphines. Other arrangements are also envisaged, such as when the metal ion is ^{99m}Tc it can be incorporated by means of ^{99m}Tc(CO)₃ radiochemistry. Examples of radiopharmaceutical precursor compounds of formula I wherein X is a chelator are described in WO 97/017092 and WO 03/093412.

In a preferred embodiment the non-radiolabelled radiopharmaceutical precursor used in the method of the first aspect is the precursor 1-(N-(t-butoxycarbonyl)amino)-3-[((trifluoromethyl)sulfonyl)oxy]-cyclobutane-1-carboxylic acid ethyl ester (FACBC triflate precursor) of formula II:

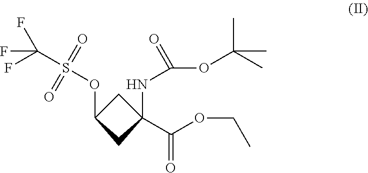 [SMILES: CCOC(=O)[C@]1(NC(=O)OC(C)(C)C)C[C@@H](OS(=O)(=O)C(F)(F)F)C1]

The compound of formula II has acceptable stability at storage conditions between −20° C. and −5° C. However, stability testing at 25° C. has shown that this compound is stable at only shorter periods, such as 1 month, and that when the degradation it then progresses very quickly. The present inventors postulated that water is likely to be the predominant cause which starts the degradation as the compound has been observed to be hygroscopic. The degraded rest compound from the testing at 25° C. is a glass like product.

In another aspect, the invention provides a method for storing a radiopharmaceutical precursor wherein said precursor is stabilised by storing said precursor in the presence of a drying agent. The suitable and preferred definitions for the radiopharmaceutical precursor and the drying agent provided for the method for improving the stability of a radiopharmaceutical precursor are applicable for the method of storing a radiopharmaceutical precursor.

Using the method of the invention, the radiopharmaceutical precursor, suitably a non-radiolabelled amino acid derivative, such as that of formula II, may be stored for extended periods of for example up to 18 months, suitably up to 6 months, more suitably for up to 8 weeks, at temperatures at or below ambient, for example at 10-35° C., suitably at 18-25° C. Storage at ambient temperature is particularly convenient.

By the use of an automated radiosynthesis system a variety of tracers can be prepared by automatically ¹⁸F-labelling of given precursors. The [¹⁸F]FACBC drug substance is prepared in the proprietary automated synthesiser unit Fastlab™, and the synthesiser module is computer-controlled. Reagents are supplied in reagent vials that are manually assembled on a cassette, preferably at the production site of the cassette, and then assembled on the synthesiser before start of the synthesis. The synthetic route for labelling of the FACBC triflate precursor to prepare [¹⁸F]FACBC is provided in the scheme below.

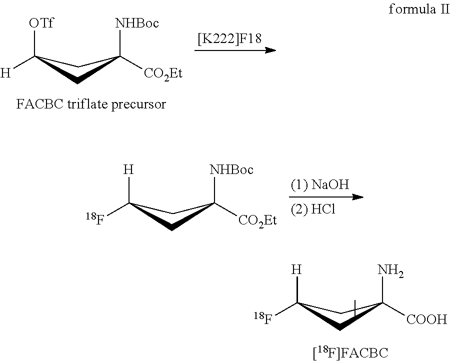

The synthesis of [¹⁸F]FACBC on the automated synthesiser unit is based on nucleophilic displacement of a triflate group (OTf) by [¹⁸F]fluoride from the precursor of formula II. [¹⁸F]Fluoride ion is typically obtained as an aqueous solution which is a product of the irradiation of an [¹⁸O]-water target. Certain steps are carried out in order to convert [¹⁸F]fluoride into a reactive nucleophilic reagent, before its use in nucleophilic radiolabelling reactions. These steps include the elimination of water from [¹⁸F]fluoride ion and the provision of a suitable counterion (Handbook of Radiopharmaceuticals 2003 Welch & Redvanly eds. Chapter 6 pp 195-227). The [¹⁸F]fluoride may for example be introduced with a solution of kryptofix (K222), potassium carbonate, water and acetonitrile into the reaction vessel. The radiofluorination reaction can then carried out using anhydrous solvents (Aigbirhio et al 1995 J Fluor Chem; 70: pp 279-87). The ¹⁸F-labelled intermediate compound then undergoes two deprotecting steps, where the ethyl and the tert-butoxy carbonyl (Boc) protecting groups are removed by basic (NaOH) and acidic (HCl) hydrolysis, respectively. Deprotection techniques are well-known to those of skill in the art. A wide range of protecting groups as well as methods for their removal is described in ‘Protective Groups in Organic Synthesis’, Theorodora W. Greene and Peter G. M. Wuts, (Fourth Edition, John Wiley & Sons, 2007).

The FACBC triflate precursor of formula II can be prepared as described in EP1978015, FIGS. 1-3, steps 1-5, and the process is hereby incorporated by reference. In summary, the first step comprises hydrolysis of syn-5-(3-benzyloxycyclobutane)hydantoin by addition of barium hydroxide Ba(OH)₂ to the solution and refluxing the mixture at 114° C. for 24 hours or longer. In the next ethyl esterification step, syn-1-amino-3-benzyloxycyclobutane-1-carboxylic acid is dissolved in ethanol and reacted with thionyl chloride to yield syn-1-amino-3-benzyloxycyclobutane-1-carboxylic acid ethyl ester. The third step comprises addition of tert-butoxycarbonyl (Boc) to the amine function by reaction of the carboxylic acid ethyl ester with tert-butyl dicarbonate (Boc)₂O, and the resultant material is purified by chromatography to obtain syn-1-(N-(t-butoxycarbonyl)amino)-3-benzyloxy-cyclobutane-1-carboxylic acid ethyl ester. The benzyl-protected intermediate is then deprotected in the next step by dissolving in ethanol, adding palladium on activated carbon and applying a small positive H₂-pressure over the reaction mixture. The resultant material is purified by chromatography to yield syn-1-(N-(t-butoxycarbonyl)amino)-3-benzyloxy-cyclobutane-1-carboxylic acid ethyl ester for use in the next step, which comprises reaction with trifluoromethanesulfonic anhydride, followed by chromatographic purification with subsequent re-crystallization of the material in order to obtain syn-1-(N-(t-butoxycarbonyl)amino)-3-[((trifluoromethyl)sulfonyl)oxy]-cyclobutane-1-carboxylic acid ethyl ester.

In the state of the art process, the radiopharmaceutical precursor, such as the compound of formula II, is stored separately from the other reagents to avoid degradation initiated by water, such as in the fridge. With the method of the invention the shelf-life of the precursor is considerably improved and the precursor can be stored at ambient temperature, for example in the same package as the other reagents or as part of a preassembled cassette. Hence, by using a drying agent stable storage conditions for the radiopharmaceutical precursor at room temperature have been found. Accordingly, one advantage with the method of the invention is that the radiopharmaceutical precursor can be assembled on the cassette, used in the automated synthesiser, during the manufacturing of the cassette. Another advantage is that the precursor can be stored at the same conditions as the cassette and the other reagents. This is convenient for both the manufacturer of the cassette and the reagents and for the user of the automated synthesiser.

In another aspect, the invention provides a composition comprising a radiopharmaceutical precursor and a drying agent. As stated above, the radiopharmaceutical precursor as used herein means a compound which may become radiolabelled, suitably with [¹⁸F] or [¹¹C] to prepare a radiolabelled PET tracer. In a preferred embodiment the radiopharmaceutical precursor is suitable for radiolabelling with [¹⁸F]. The precursor is preferably a cyclobutane based amino acid derivative, and more preferably, the radiopharmaceutical precursor is a compound of formula I, as provided for the first aspect. Most preferably, the radiopharmaceutical precursor is a compound of formula II, i.e. 1-(N-(t-butoxycarbonyl)amino)-3-[((trifluoromethyl)sulfonyl)oxy]-cyclobutane-1-carboxylic acid ethyl ester (FACBC triflate precursor). As for the first aspect, the drying agent can be selected from different inert substances and is preferably a molecular sieve or silica gel. The composition comprising the precursor and the drying agent is a formulation wherein the precursor is packed in the drying agent such that the drying agent removes excessive humidity from all the precursor material. The two components are mixed together such that all the precursor material is in close vicinity to the drying agent, i.e. it surrounds or is surrounded by the drying agent. In a preferred embodiment, a composition of the precursor and the drying agent is stored in a container, such as a vessel or a cartridge that can be assembled on a cassette for an automated synthesiser. The composition of the precursor and the drying agent may be housed in a disposable or removable cassette designed for use with an automated synthesis apparatus.

Therefore, in a further aspect the invention provides a cassette for an automated synthesis apparatus comprising a composition of radiopharmaceutical precursor and a drying agent. As demonstrated herein, the improved stability of the radiopharmaceutical precursor when stored as such composition means that the cassette can be provided complete with all the reagents required for the radiolabelling reaction, except for the radioisotope, and the cassette can be stored at ambient temperature thus avoiding the need for refrigeration.

Where the cassette of the present invention is for the preparation of an ¹⁸F-labelled PET radiopharmaceutical it comprises:
(i) a vessel containing a composition of the radiopharmaceutical precursor and the drying agent as defined herein; and
(ii) means for eluting the vessel with a suitable source of [¹⁸F]-fluoride as defined herein.

The cassette may also comprise an ion-exchange cartridge for removal of excess [¹⁸F]-fluoride.

Where the ¹⁸F-labelled PET radiopharmaceutical is an ¹⁸F-labelled cyclobutane-based amino acid derivative, the radiopharmaceutical precursor is a compound of formula I as suitably and preferably defined herein. The cassette in this case may also comprise a cartridge for deprotection of the compound following the ¹⁸F labelling reaction.

In yet another aspect, the invention provides a process for producing a radiolabelled radiopharmaceutical, the process includes a step of reacting a radioactive fluorine to a radiopharmaceutical precursor, such as that of formula I or II, wherein the precursor is stabilized by being in the presence of a drying agent.

In such process, a composition comprising a radiopharmaceutical precursor and a drying agent is used, and the process preferably includes a step of washing the composition with a solvent, to dissolve the precursor and transfer it to a reaction vessel for labelling with the radioactive fluorine source. The drying agent will remain in the container wherein the composition of the precursor and the drying agent was stored prior to reaction with the radioactive fluorine. In a preferred embodiment, the processes take place using an automated synthesizer and the radiopharmaceutical precursor is stored in a container, such as in a cartridge, on a cassette that fits into the synthesizer.

In a particularly preferred embodiment the [¹⁸F]FACBC drug substance is prepared in a process on an automated synthesizer including a cassette wherein reagents are supplied in different containers, and wherein the FACBC triflate precursor of formula II is provided in one container in a composition with a drying agent. The stabilized composition of FACBC triflate precursor mixed with a drying agent is preferably washed with acetonitrile, the FACBC triflate precursor is solved and transported to a reaction vessel where it is reacted with a radioactive fluorine. Then the ¹⁸F-labelled intermediate compound undergoes two deprotecting steps, where the ethyl and the Boc protecting groups are removed by basic and acidic hydrolysis, respectively.

As the precursor of formula II is expensive it is important to avoid waste of this, and all the precursor material should preferably be washed out of the container for reaction with the radiolabel. In a preferred embodiment a small cartridge is used for storing the composition comprising the drying agent and the precursor. To ensure that the cartridge can be completely emptied the cartridge is preferably positioned such that it has the outlet opening at the bottom, i.e. pointing downwards, in the cassette.

The suitable and preferred definitions of the radiopharmaceutical precursor and the drying agent provided above for the methods of the invention are equally applicable for the cassette and process of the invention.

The invention is illustrated by way of the following non-limiting example.

### EXAMPLE 1

### Stability of 1-(N-(t-butoxycarbonyl)amino)-3-((trifluoromethyl)sulfonyl)oxy-cyclobutane-1-carboxylic acid ethyl ester (formula II) when Packed with a Drying Agent

To test the stability of the precursor of formula II when packed with a drying agent, a sample of the precursor was analysed by ¹⁹F and ¹H measurements.

The following drying agents were used:

Molecular sieve: 0.4 nm, Merck, Art. 5708, beads about 2 mm, pore size 4A
Silica: Kiselgel, Merck, product no. 1.01969.5000

The drying agents used in the tests are typical drying agents for removing water from solvents and desiccators. The drying agents were both dried at 120° C. for about 90 hours before use.

The storage conditions for the precursor together with a drying agent were tested by storing together in the bottom of a small sample glass (mixed together) placed on a shelf in the lab (i.e. at room temperature). The amount of precursor and drying agent mixed together is given in the Table 1. Also some samples without drying agent were set up. The first analysis of the samples with solid product was performed after 3.5 months storage and a second analysis was carried out after 5 months storage.

TABLE 1

| | | | ¹⁹F NMR |
|---|---|---|---|
| | | ¹⁹F NMR results, | results, |
| | | degradation | degradation |
| | Weight of | (mole %) | (mole %) |
| Sample | PRECURSOR/ | after | after |
| combination | drying agent | 3.5 months | 5 months |
| PRECURSOR + | 52.5 mg/212.2 mg | 1.3 | 8.2 |
| Molecular sieve | | | |
| PRECURSOR + | 50.3 mg/200.8 mg | 1.4 | 4.6 |
| Silica | | | |
| PRECURSOR, | 54.1 mg | 30.7 | 96.6 |
| stored without | | | |
| drying agents | | | |
| PRECURSOR | 50.3 mg | 0.7 | 0.6 |
| stored in freezer, | | | |
| reference | | | |

The results show that putting drying agents together with the precursor has an effect on the stability of the precursor, and this is considerably increased. These results show that the sample of the precursor without a drying agent gives a degradation of about 30 mole % after 3.5 months, and about 97 mole % after 5 months, compared to the two samples wherein the precursor is mixed with drying agents according to the invention giving about 1.4 mole % degradation after 3.5 months and 8.2 and 4.6 mole % degradation only after 5 months.

1. A method for improving the stability of a radiopharmaceutical precursor comprising storing said radiopharmaceutical precursor in the presence of a drying agent wherein said radiopharmaceutical precursor is a compound of formula I: 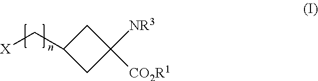
wherein:
n is an integer of 0 or of 1 to 4;
R¹ represents a protective group for the carboxylic acid function of the amino acid;
X is a moiety suitable for nucleophilic substitution and,
R³ is a protective group for the amino function of the amino acid.

2. (canceled)

3. (canceled)

4. A method as claimed in claim 1 wherein the radiopharmaceutical precursor is the compound of formula II:

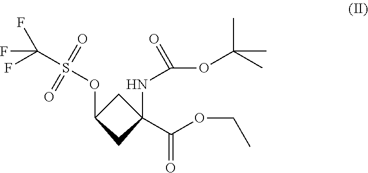 [SMILES: CCOC(=O)[C@]1(NC(=O)OC(C)(C)C)C[C@@H](OS(=O)(=O)C(F)(F)F)C1]

5. A method as claimed in claim 1 wherein the drying agent is either a molecular sieve or silica gel.

6. A composition comprising a radiopharmaceutical precursor as defined in claim 1 and a drying agent as defined in claim 1 wherein said composition is stored in a container.

7. A composition as claimed in claim 6 wherein the weight ratio between the drying agent and the precursor is between 12:1 and 1:1.

8. A cassette for an automated synthesis apparatus comprising a composition of a radiopharmaceutical precursor and a drying agent wherein said composition is as defined in claim 6.

9. A process for producing a radiopharmaceutical, wherein radioactive fluorine is reacted with a radiopharmaceutical precursor, wherein said precursor is stabilised by being in the presence of a drying agent.
